(19) United States
(12) Patent Application Publication
(10) Pub. No.: US 2023/0143593 A1
(43) Pub. Date: May 11, 2023

(54) DIGITAL PATHOLOGY RECORDS DATABASE MANAGEMENT

(71) Applicant: MEMORIAL SLOAN KETTERING CANCER CENTER, New York, NY (US)

(72) Inventors: Thomas Fuchs, New York, NY (US); Luke Geneslaw, New York, NY (US); Dig Vijay Kumar Yarlagadda, New York, NY (US)

(21) Appl. No.: 17/911,093

(22) PCT Filed: Mar. 15, 2021
(86) PCT No.: PCT/US2021/022326

§ 371 Date: Sep. 12, 2022

Related U.S. Application Data
(60) Provisional application No. 62/990,393, filed on Mar. 16, 2020.

(51) Int. Cl. G06F 21/62; G16H 10/60; G16H 30/40

(52) U.S. Cl. CPC G06F 21/6254; G16H 10/60; G16H 30/40

(57) ABSTRACT

The present disclosure is directed to systems and methods of maintaining databases of biomedical images. A server may aggregate digital pathology records from data sources onto a database. Each record may be generated by a data source using a format, and may identify a biomedical image of a sample and data identifying a subject from which the sample is obtained. The server may receive, from a client device, a query identifying a criterion. The server may access the database to identify a subset of records using the criterion. For each record of the subset, the server may identify a data source that generated the record. The server may select a de-identification policy to apply based on the data source. The server may modify the data in the record according to the de-identification policy and the format. The server may provide, to the client device, the de-identified record.

### CROSS REFERENCE TO RELATED APPLICATIONS

The present application claims priority to U.S. Provisional Application No. 62/990,393, titled “DIGITAL PATHOLOGY RECORDS DATABASE MANAGEMENT,” filed Mar. 16, 2020, which is incorporated herein by reference in its entirety.

### BACKGROUND

A biological sample may be obtained from a specimen or subject in a controlled environment, and an image of the biological sample may be acquired. Various data on the biological sample itself and the image may be compiled, collected, and evaluated in accordance with various bioinformatics techniques.

### SUMMARY

Each digital pathology record may identify or include an image of a biological sample (e.g., a whole slide image (WSI) of a tissue sample) along with metadata identifying a subject from which the sample was obtained and other information on the subject or the sample. The image of the biological sample may be generated using an imaging device (e.g., a microscopy camera) and the metadata may be generated via input (e.g., by a clinician) on a computing device. The image may be very large (e.g., greater than 500 megabytes), and may be referenced in the digital pathology record using an address (e.g., a Uniform Resource Locator (URL) or a file pathname). The metadata may, for example, include: an accession identifier; an accession date; a specimen classification; a part type; a part instance; a part description; a block instance; a block designator label; a medical record number (MRN); a slide image identifier; a scan date; a stain type; synoptic data; and final diagnosis, among others.

Individual vendors (e.g., Aperio™, Hamamatsu™, and 3DHISTECH™) or other entities may generate such digital pathology records according to a proprietary or otherwise particular format of the vendor. For example, one vendor may insert the metadata onto the image of the biological sample itself so that the metadata is visible to the user of the image. Another vendor may encode and embed the metadata on a particular set of bytes in an image file for the image of the biological sample. Another vendor may include the metadata on a separate file (e.g., a text file) in a structured or unstructured manner. In addition, these vendors may store and maintain the digital pathology records on one or more databases particular to the vendor.

Before the records are to be shared and communicated, at least a portion of the metadata may be removed or modified to obfuscate or de-identify the identity of the subject and other details regarding the acquisition of the image of the biological sample from the subject. The de-identification may be carried out in accordance with data privacy policies on protected health information (e.g., Health Insurance Portability and Accountability Act (HIPAA) privacy rules). Since different vendors may use different formats to generate and maintain such records, how the metadata in the digital pathology records is to be obfuscated may differ from vendor to vendor. As such, accessing and sharing records from a multitude of vendors in a networked environment may be difficult and cumbersome to implement due to the different number of formats in the generating and maintaining such digital pathology records by each vendor.

One approach at accounting for some of these technical challenges may be to use available vendor-specific scripts to de-identify and obfuscate the metadata in the particular digital pathology record. However, such scripts may be to de-identify records from the particular vendor, and may be incompatible with records from other vendors Another approach may include using an application for detecting and redacting the protected health information in the metadata from unstructured text files. But the utility of such applications may be limited to text files containing the metadata in unstructured format, and may not be able to remove such protected health information in records with metadata in other formats. In addition, both approaches may be inefficiently and, consume a significant amount of computing resources with no guarantee of redacting the protected information from all the records. Furthermore, these scripts may do little at addressing the sheer large size of biomedical images in such digital pathology records.

To address these and other challenges related to digital pathology records, a record service may aggregate digital pathology records from the various vendors to provide data for pathology research. The record service may have a database (e.g., a Structured Query Language (SQL) server) and a backend server with an application to handle queries (e.g., a Python™ application running on a physical Linux™ server). The database of the record service may connect with the databases associated with the vendors to pull the digital pathology records from time to time (e.g., nightly). The database in turn may store and maintain the records without performing any de-identification.

The application on the server of the records service may receive and process a query for records is received from a user (e.g., a computing device operated by a researcher). The query may include criteria (e.g., keywords, parameters, or other values) for types of digital pathology records to retrieve from the database. Using the query, the application may identify the records in the database that satisfy the criteria of the query (such records may be also referred herein as a cohort). For each record found from the database, the application may identify a vendor that generated the record and may select a de-identification policy for the record based on specification of the vendor. The de-identification policy for the vendor may indicate a location of the metadata types in the record (e.g., in a particular byte in the image file, an area within the image, or a separate text file). The de-identification policy may also specify an operation (e.g., deletion, truncation, or replacement) to obfuscate the protected information in the metadata at the location. In accordance with the selected policy, the application on the server may modify the metadata in the digital pathology record found using the query. Once the metadata are modified, the application may provide the de-identified records to the user that requested for the records.

With the de-identification of the digital pathology records, the application may store and maintain the de-identified versions of the records onto the database. The application may link or associate the de-identified and original versions of the digital pathology records on the database. In this manner, the application may provide capabilities for querying the database to select a cohort of digital pathology records and create de-identified datasets for the cohort. The record provided by the record service may include discrete pathology report data that has been de-identified and the biomedical image associated with the report. With each record having very large image files (e.g., over 500 megabytes), it may be infeasible to de-identify every record as the records are received from the vendors. By performing de-identification on the digital pathology records found using the query, the data service may avoid the issue of impracticability in de-identifying every record, thereby saving consumption of computational resources.

At least one aspect of the present disclosure is directed to a method of maintaining databases of biomedical images. One or more processors may aggregate a plurality of digital pathology records from a plurality of data sources onto a database. Each of the plurality of digital pathology records may be generated by a data source of the plurality of data sources in accordance with a format used by the data source. Each of the plurality of digital pathology records may identify a biomedical image of a sample and data identifying a subject from which the sample is obtained. The one or more processors may receive, from a client device, a query identifying a selection criterion for retrieving digital pathology records from the database. The one or more processors may access the database to identify a subset of digital pathology records from the plurality of digital pathology records using the selection criterion identified by the query. For each digital pathology record of the subset, the one or more processors may identify a data source of the plurality of data source that generated the digital pathology record. The one or more processors may select, from a plurality of de-identification policies, a de-identification policy to apply to the digital pathology record based on the data source. The one or more processors may modify the data identifying the subject from the digital pathology record in accordance with the selected de-identification policy and the format used by the data source to obtain a de-identified digital pathology record. The one or more processors may provide, to the client device, the de-identified digital pathology record in response to modifying the data identified the subject.

In some embodiments, the one or more processors may identify, for each digital pathology record of the subset, in accordance with the de-identification policy, the data to be modified in the digital pathology record, the de-identification specifying at least one of a truncation, a removal, or an overwrite of at least a corresponding portion of the data.

In some embodiments, for at least one digital pathology record of the subset, the one or more processors may identify, using pattern recognition, additional information to modify from the digital pathology record subsequent to modifying the data in accordance with the de-identification policy. In some embodiments, the one or more processors may modify the additional information in the digital pathology record to obtain the de-identified digital pathology record.

In some embodiments, for at least one digital pathology record of the subset, the one or more processors may identify a first file containing the data and a second file containing the biomedical image for the digital pathology record in accordance with the format used by the data source to generate the digital pathology record. In some embodiments, modifying the data may include modifying the data contained in the first file separate from the second file in accordance with the de-identification policy.

In some embodiments, for at least one digital pathology record of the subset, the one or more processors may identify a file including a first portion corresponding to the data and one or more second portions corresponding to the biomedical image for the digital pathology record in accordance with the format used by the data source to generate the digital pathology record. In some embodiments, modifying the data may include modifying the data in the first portion of the file for the digital pathology record of the subset in accordance with the de-identification policy

In some embodiments, aggregating the plurality of digital pathology records may include aggregating a plurality of location identifiers from the plurality of data sources. The plurality of location identifiers may identify the biomedical image and the data for each of the plurality of digital pathology records. In some embodiments, accessing the database may include retrieving the subset of digital pathology records from one or more of the plurality of data sources using a subset of location identifiers corresponding to the subset of digital pathology records.

In some embodiments, accessing the database may include accessing the database to identify the subset of digital pathology records from the plurality of digital pathology records. Each of the subset of digital pathology records may have an indication of permission for use. In some embodiments, aggregating the plurality of digital pathology records may include maintaining the plurality of digital pathology records retrieved from the plurality of data sources, without removal of the data identifying the subject in each of the plurality of digital pathology records prior to receiving the query.

In some embodiments, aggregating the plurality of digital pathology records may include aggregating the plurality of digital pathology records, each of the plurality of digital pathology records identifying the data identifying a date at which the biomedical image of the sample from the subject is acquired, a part description, an image identifier, and a descriptor. In some embodiments, the one or more processors may store, for each digital pathology record of the subject, the de-identified digital pathology record onto the database to replace the corresponding digital pathology record of the subject.

At least one aspect of the present disclosure is directed to a system for maintaining databases of biomedical images. The system may include one or more processors coupled with memory. The one or more processors may aggregate a plurality of digital pathology records from a plurality of data sources onto a database. Each of the plurality of digital pathology records may be generated by a data source of the plurality of data sources in accordance with a format used by the data source. Each of the plurality of digital pathology records may identify a biomedical image of a sample and data identifying a subject from which the sample is obtained. The one or more processors may receive, from a client device, a query identifying a selection criterion for retrieving digital pathology records from the database. The one or more processors may access the database to identify a subset of digital pathology records from the plurality of digital pathology records using the selection criterion identified by the query. For each digital pathology record of the subset, the one or more processors may identify a data source of the plurality of data source that generated the digital pathology record. The one or more processors may select, from a plurality of de-identification policies, a de-identification policy to apply to the digital pathology record based on the data source. The one or more processors may modify the data identifying the subject from the digital pathology record in accordance with the selected de-identification policy and the format used by the data source to obtain a de-identified digital pathology record. The one or more processors may provide, to the client device, the de-identified digital pathology record in response to modifying the data identified the subject.

In some embodiments, the one or more processors may identify, for each digital pathology record of the subset, in accordance with the de-identification policy, the data to be modified in the digital pathology record, the de-identification specifying at least one of a truncation, a removal, or an overwrite of at least a corresponding portion of the data.

In some embodiments, for at least one digital pathology record of the subset, the one or more processors may identify, using pattern recognition, additional information to modify from the digital pathology record subsequent to modifying the data in accordance with the de-identification policy. In some embodiments, the one or more processors may modify the additional information in the digital pathology record to obtain the de-identified digital pathology record.

In some embodiments, for at least one digital pathology record of the subset, the one or more processors may identify a first file containing the data and a second file containing the biomedical image for the digital pathology record in accordance with the format used by the data source to generate the digital pathology record. In some embodiments, the one or more processors may modify the data contained in the first file separate from the second file in accordance with the de-identification policy.

In some embodiments, for at least one digital pathology record of the subset, the one or more processors may identify a file including a first portion corresponding to the data and one or more second portions corresponding to the biomedical image for the digital pathology record in accordance with the format used by the data source to generate the digital pathology record. In some embodiments, the one or more processors may modify the data in the first portion of the file for the digital pathology record of the subset in accordance with the de-identification policy

In some embodiments, the one or more processors may aggregate a plurality of location identifiers from the plurality of data sources. The plurality of location identifiers may identify the biomedical image and the data for each of the plurality of digital pathology records. In some embodiments, the one or more processors may retrieve the subset of digital pathology records from one or more of the plurality of data sources using a subset of location identifiers corresponding to the subset of digital pathology records.

In some embodiments, the one or more processors may access the database to identify the subset of digital pathology records from the plurality of digital pathology records. Each of the subset of digital pathology records may have an indication of permission for use. In some embodiments, the one or more processors may maintain the plurality of digital pathology records retrieved from the plurality of data sources, without removal of the data identifying the subject in each of the plurality of digital pathology records prior to receiving the query.

In some embodiments, the one or more processors may aggregate the plurality of digital pathology records, each of the plurality of digital pathology records identifying the data identifying a date at which the biomedical image of the sample from the subject is acquired, a part description, an image identifier, and a descriptor. In some embodiments, the one or more processors may store, for each digital pathology record of the subject, the de-identified digital pathology record onto the database to replace the corresponding digital pathology record of the subject.

### BRIEF DESCRIPTION OF THE DRAWINGS

The objects, aspects, features, and advantages of the disclosure will become more apparent and better understood by referring to the following description taken in conjunction with the accompanying drawing, in which:

FIG. 1 is a block diagram of a system maintaining databases of biomedical images in accordance with an illustrative embodiment;

FIG. 2 is a sequence diagram of a process for maintaining databases of biomedical images in accordance with an illustrative embodiment;

FIG. 3 is a sequence diagram of a process for maintaining databases of biomedical images in accordance with an illustrative embodiment;

FIG. 4 is a sequence diagram of a process for maintaining databases of biomedical images in accordance with an illustrative embodiment;

FIG. 5 is a flow diagram of a method of maintaining databases of biomedical images in accordance with an illustrative embodiment; and

FIG. 6 is a block diagram of a server system and a client computer system in accordance with an illustrative embodiment.

The drawings are not necessarily to scale; in some instances, various aspects of the subject matter disclosed herein may be shown exaggerated or enlarged in the drawings to facilitate an understanding of different features. In the drawings, like reference characters generally refer to like features (e.g., functionally similar and/or structurally similar elements).

### DETAILED DESCRIPTION

Following below are more detailed descriptions of various concepts related to, and embodiments of, systems and methods for maintaining databases of biomedical images. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes systems and methods of maintaining databases of biomedical images; and

Section B describes a network environment and computing environment which may be useful for practicing various embodiments described herein.

### A. Systems and Method of Maintaining Databases of Biomedical Images

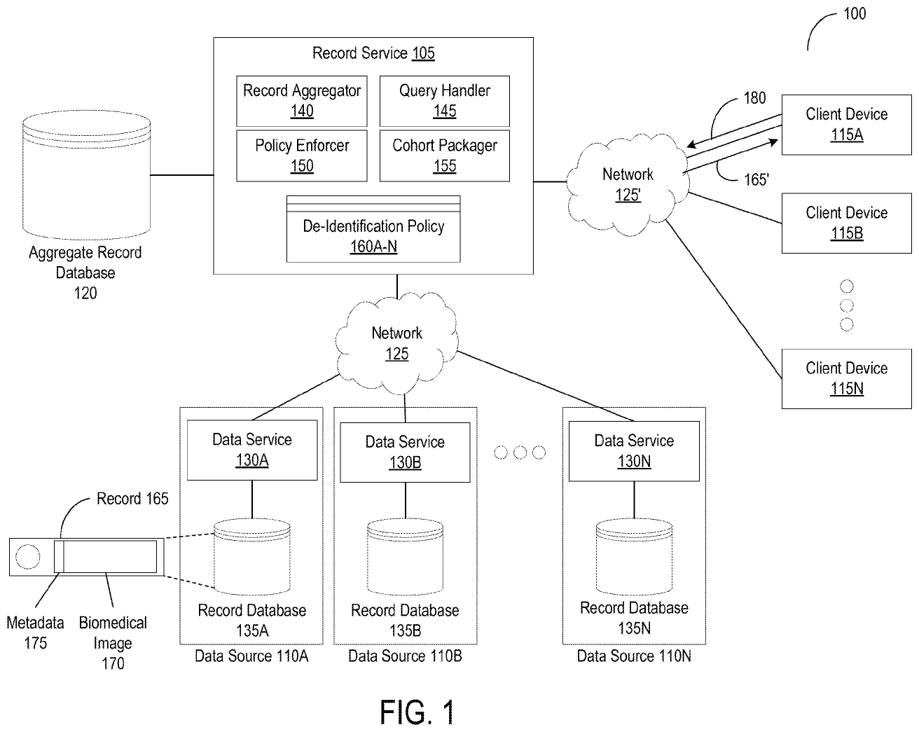

Referring now to FIG. 1, depicted is a block diagram of an environment or a system 100 maintaining databases of biomedical images in digital pathology records. In overview, the system 100 may include at least one record service 105, one or more data sources 110A-N (hereinafter generally referred to as a data source 110), one or more client devices 115A-N (hereinafter generally referred to as a client device 115), and one or more networks 125 or 125′ among others. The data source 110 may include or may be formed by at least one data service 130A-N (hereinafter generally referred to as a data service 130) and at least one record database 135A-N (hereinafter generally referred to a record database 135) communicatively coupled to one another. The record service 105 may include at least one aggregate record database 120 (sometimes generally referred herein as a record database), at least one record aggregator 140, at least one query handler 145, at least one policy enforcer 150, at least one cohort packager 155, and one or more de-identification policies 160A-N (hereinafter generally referred to as a de-identification policy 160), among others. Each of the modules, units, or components in system 100 (such as the record service 105 and its components, each data source 110 and its components, the client devices 115, and the networks 125 and 125′) may be implemented using hardware or a combination of hardware and software as detailed herein in Section B.

At each data source 110, the data service 130 may maintain and manage the record database 135 in storing one or more digital pathology records 165 (hereinafter generally referred to as record 165). Each data source 110 may be operated and administered by a respective vendor of bioinformatics data for histopathology, and may have a particular format (e.g., a proprietary protocol or standard) to package and maintain the bioinformatics data on the database 135. For example, the format used by the vendor of the first data source 110A may differ from the format used by the vendor of the second data source 110B. Each record 165 on the database 135 may be generated, maintained, stored, and indexed in accordance with the format of the data source 110 to which the record database 135 belong. Generally, across different vendors (and by extension the associated data sources 110 and databases 135), each record 165 may include at least one biomedical image 170 and metadata 175 associated with the biomedical image 170.

To generate the record 165, the data service 130 may identify the biomedical image 170. The biomedical image 170 may be acquired via an imaging device from a biological sample of a subject for histopathology. For instance, a microscopy camera may acquire the biomedical image 170 of a histological section corresponding to a tissue sample obtained from an organ of a human subject on a glass slide stained using hematoxylin and eosin (H&E stain). The subject for the biomedical image 170 may include, for example, a human, an animal, a plant, or a cellular organism, among others. The biological sample may be from any part (e.g., anatomical location) of the subject, such as a muscle tissue, a connective tissue, an epithelial tissue, or a nervous tissue in the case of a human or animal subject. The imaging device used to acquire the biomedical image 170 may include an optical microscope, a confocal microscope, a fluorescence microscope, a phosphorescence microscope, or an electron microscope, among others Upon acquisition, the imaging device may send, relay, or otherwise provide the biomedical image 170 to the data service 130.

The data service 130 may receive the biomedical image 170 from the imaging device (or a computing device communicatively coupled with the imaging device). The biomedical image 170 may correspond to an image file or a set of image files forming the entire image of the biological sample. For example, the set of image files generated for the biomedical image 170 may be in accordance with the Digital Imaging and Communications in Medicine (DICOM) standard. The one or more image files constituting the biomedical image 170 may have a relatively large size, ranging from 500 megabytes to 50 gigabytes in total. In some embodiments, the data service 130 may perform one or more pre-processing operations on the biomedical image 170 to standardize or regularize for storage onto the record database 135. The pre-processing operations may include, for example, resizing, de-noising, segmentation, or decompression, among others. Upon receipt from the imaging device, the data service 130 may store and maintain the biomedical image 170 on the record database 135.

In conjunction, the data service 130 may identify the metadata 175 associated with the biomedical image 170. The metadata 175 may include, assign, or otherwise identify one or more characteristics regarding the subject from which the biological sample for the biomedical image 170 is acquired and regarding the acquisition of the biomedical image 170 from the subject. The metadata 175 may be generated via one or more inputs on a computing device and may be received from the computing device. For example, a clinician evaluating the subject and the tissue sample from which the biomedical image 170 is obtained may interact with a graphical user interface presented on the computing device to enter values for the metadata 175. The metadata 175 may include one or more fields and values associated with each field. The one or more fields of the metadata 175 may include, for example:
An accession identifier referencing an indication of permission (e.g., agreement) by the subject in providing the biological sample for the biomedical image 170;
An accession date corresponding a year, month, day, or time of the indication of the permission by the subject;
A part type identifying an anatomical location (e.g., type of tissue, a type of organ, or other part of body) from which the biological sample for the biomedical image 170 is obtained;
A specimen class identifying a category of tissue or retrieval mechanism (e.g., surgical pathology tissue, department consult, cytology tissue, etc.);
A part instance identifying the order a particular specimen part was accessioned in a particular case;
A part description identifying a brief textual description of the tissue specimen;
A block instance identifying the order a particular specimen block was accessioned in a particular case;
A block designator label identifying a brief textual code or description of a particular paraffin block in a particular case;
A medical record number used by the vendor operating the data service 130 to reference the subject from which the biological sample for the biomedical image 170 is obtained;
A slide image identifier referencing the biomedical image 170 acquired of the biological sample from the subject;
A scanning date indicating a year, month, day, or time at which the biomedical image 170 is acquired from the biological sample of the subject;
A stain type identifying a type of stain (hematoxylin and eosin (H&E) stain, hemosiderin stain, a Sudan stain, a Schiff stain, a Congo red stain, a Gram stain, a Ziehl-Neelsen stain, a Auramine—rhodamine stain, a trichrome stain, a Silver stain, and Wright's Stain, among others) used to stain the biological sample when the biomedical image 170 is acquired;
Synoptic data identifying discrete diagnostic data entered into a case by a pathologist using a particular predefined synoptic worksheet template (e.g., a worksheet for prostate needle core biopsy with a predefined field for Gleason grade=8);
Subject traits identifying characteristics (e.g., age, race, gender, and geographical location) of the subject from which the biological sample for the biomedical image 170 is obtained; and
Final diagnosis descriptor identifying a condition attributed to the biological sample for the biomedical image 170.

Each of the fields in the metadata 175 may have or be associated with one or more values entered via the computing device. With the entry of the values, the computing device may send the metadata 175 in addition to the biomedical image 170 to the data service 130. The data service 130 may receive the metadata 175 from the computing device. Upon receipt from the computing device, the data service 130 may store and maintain the metadata 175 for the associated biomedical image 170 on the record database 135. The one or more biomedical image metadata fields may include, for example:
Magnification level identifying the optical zoom level used by the imaging device at the time of scanning;
Scan instance identifying the order in which a particular biomedical image was scanned relative to other scans of the same physical sample;
Scan time identifying the duration the imaging device took to complete the biomedical image scan;
Scanner brand and model identifying the type of imaging device used to scan a biomedical image; and
Tissue size identifying the physical dimensions of the tissue detected by the imaging device

Using the biomedical image 170 and the associated metadata 175, the data service 130 may generate the record 165. The use of the biomedical image 170 and the metadata 175 to form, package, or generate the record 165 may be in accordance with the format for the data source 110 to which the data service 130 belongs. The format may include, indicate, or specify a template or a set of operations to be applied by the data service 130 to the biomedical image 170 and the metadata 175 in generating the record 165. The template or the set of operations to be applied may be configured by the vendor or entity associated with the data source 110 to which the data service 130 belongs to. The template may correspond to or include one or more container files each with one or more elements to include or identify the biomedical image 170 and the metadata 175. For example, the template may include a space in a file for a location identifier (e.g., a Uniform Resource Locator (URL) or a file pathname) of the image file for the biomedical image 170 and one or more spaces for the fields and values of the metadata 175. The set of operations may enumerate or specify processes to apply to the biomedical image 170 and the metadata 175 in generating the record 165.

The formats may differ among the various data sources 110 associated with the data services 130. For example, the processes of the format configured by the vendor associated with the first data source 110A may differ from those specified by the vendor associated with the second data source 110B. In some embodiments, the format for the data source 110 may specify a combination (e.g., embedding) of the biomedical image 170 and the metadata 175 in generating the record 165. For example, the format may specify that the metadata 175 are to be inserted into at least one or more specified bytes in one or more image files constituting the biomedical image 170. The insertion of the bytes into the image files may keep the visual appearance of the biomedical image 170 unaltered. The format for the data source 110 may also specify that the metadata 175 are to be inserted onto one or more portions of the biomedical image 170 itself so that fields or values of the metadata 175 are visible on the biomedical image 170. In some embodiments, the format for the data source 110 may specify that a union of the biomedical image 170 and the metadata 175 in generating the record 165. For example, the format may specify that the metadata 175 are to be stored on one or more files (e.g., text files in a comma separated value (CSV) format) separated from the image files for the biomedical image 170. The format may also specify that a location identifier (e.g., a Uniform Resource Locator (URL) or a file pathname) referencing the biomedical image 170 is to be included in the text file containing the fields and values of the metadata 175.

By applying the format to the biomedical image 170 and the metadata 175, the data service 130 may generate the record 165. In some embodiments, the data service 130 may identify the metadata 175 associated with the biomedical image 170. For example, the data service 130 may find the biomedical image 170 on the record database 135 with the same identifier as the scan image identifier listed in the metadata 175. Upon identification, the data service 130 may combine or unite the biomedical image 170 with the metadata 175 in accordance with the specifications of the format for the data source 110 to generate the record 165. For example, the data service 130 may parse the image files for the biomedical image 170 to identify one or more bytes to insert the fields and values of the metadata 175, and may generate the biomedical image 170 with the embedded metadata 175 as the record 165. In another example, the data service 130 may create a separate text file for the metadata 175 and package the text file for the metadata 175 and the image files for the biomedical image 170 to generate the record 165. In this example, the text file and the image files may constitute the record 165. With the generation and packaging of the record 165, the data service 130 may store and maintain the record 165 on the record database 135. The data service 130 may repeat the process of generating, storing, and maintain records 165 on the record database 135 using other biomedical images 170 and associated metadata 175. In addition, the data service 130 may make available (e.g., to the record service 105 and the client devices 115) for access the records 165 stored and maintained on the record database 135.

The record aggregator 140 running on the record service 105 may collect, gather, or otherwise aggregate the records 165 from record databases 135 from multiple data sources 110 onto the aggregate record database 120. To aggregate, the record aggregator 140 may establish communications with each data source 110 via the network 125. The communications may include, for example, a secure communications session with the data service 130 or the record database 135 of the data source 110 over the network 125. With the establishment of the communications, the record aggregator 140 may access the data source 110 (or the associated data service 130 or the record database 135) to identify and retrieve the records 165 maintained by the data source 110. In some embodiments, the record aggregator 140 may retrieve the records 165 from the data source 110 in accordance with a schedule. The schedule may indicate a range of times (e.g., a time of day) during which the record aggregator 140 is to access the record database 135 and retrieve the records 165 from the data source 110. For example, the record aggregator 140 may maintain a timer to keep track of time, and may access the record database 135 of the data source 110 when the time is between 2:00 am and 4:00 am as specified by the schedule to pull the records 165.

With retrieval from each data source 110, the record aggregator 140 may store and maintain the records 165 on the aggregate record database 120. The storage and maintenance of the records 165 may be performed by the record aggregator 140 without removal of any portion of the metadata 175 in each record 165. In some embodiments, the record aggregator 140 may generate or include a label identifying the data source 110 from which the record 165 originates to store with the record 165 on the aggregate record database 120. In some embodiments, the record aggregator 140 may store and maintain the location identifier for the biomedical image 170 in each record 165. For example, rather than storing the one or more image files forming the biomedical image 170 in the record 165, the record aggregator 140 may maintain links (e.g., URLs) to the image files. The record 165 itself may also contain the links to the image files as opposed to the image files themselves. In some embodiments, the record aggregator 140 may store and maintain the image files forming the biomedical image 170 in the record 165 along with the metadata 175. For example, the record aggregator 140 may store the record 165 including the image files for the biomedical image 170 with the metadata 175 as separate file or embedded into the image files onto the aggregate record database 120. Upon storage, the record aggregator 140 may make available for access the records 165 on the aggregate record database 120. The maintenance and accessing of the records 165 on the aggregate record database 120 may be in accordance with a relational database management (RDBM) protocol, such as Structured Query Language (SQL), JavaScript Database Connectivity (JDBC), Open Database Connectivity (ODBC), or Apache database architectures, among others.

The client device 115 may communicate with the record service 105 over the network 125 or 125′. The client device 115 may be operated by a user (e.g., a researcher) or another entity intending to view biomedical images 170 of biological samples as part of a histopathological study. In some embodiments, the client device 115 may establish communications with the record service 105 via the network 125′. The communications may include, for example, a secure communications session between the record service 105 and the client device 115. The secure communications session may be established upon provision by the client device 115 of proper account identifier and authentication credentials to the record service 105. The network 125′ between the record service 105 and the client device 115 may differ or may be separate from the network 125 among the record service 105 and the one or more data sources 110. The separation of the networks 125 and 125′ may be to prevent the client device 115 from direct accessing of the records 165 on the records 165 maintained by the data sources 110.

With the establishment of communications, the client device 115 may transmit or send at least one query 180 (sometimes referred herein as a request) to the record service 105 via the network 125 or 125′ for retrieval of records 165. In some embodiments, the generation and sending of the query 180 by the client device 115 may be in accordance with the same RDBM protocol used by the record service 105. The query 180 may include one or more criteria for selection and retrieval of records 165 from the aggregate record database 120. The criteria of the query 180 may include, for example: one or more specimen classes corresponding to anatomical locations from which the biological sample is obtained; a scanning timeframe identifying a range of times during which the biomedical image 170 of the sample is acquired; stain types identifying types of stain used to treat the biological sample; traits of the subject from which the biological sample is obtained, condition diagnosed for the biological sample, and a number of records 165 to retrieve, among others. In some embodiments, the criteria may correspond to one or more keywords or phrases in the query 180. In some embodiments, the criteria may correspond to one or more selections on a user interface of an application running on the client device 115 for accessing the record service 105. For example, a researcher seeking records 165 on breast cancer whole slide images (WSIs) may click on the corresponding checkboxes on a graphical user interface to generate the query 180 to send to the record service 105. Upon generation, the client device 115 may send the query 180 to the record service 105 to retrieve records 165 from the aggregate record database 120 via the network 125 or 125′.

The query handler 145 running on the record service 105 may receive the query 180 sent by the client device 115. Upon receipt, the query handler 145 may parse the query 180 to identify one or more criteria for selecting or retrieving records 165 from the aggregate record database 120. The receipt and parsing of the query 180 may be separate or in conjunction to the aggregation of the records 165. In some embodiments, the parsing of the query 180 may be in accordance with the relational database management protocol. In some embodiments, the query handler 145 may apply one or more natural language processing (NPL) algorithms on the keywords in the query 180 to identify the selection criteria for retrieving records 165. The NPL algorithms may include lemmatization, sentence structure extraction, information extraction, stemming, named entity recognition (NER), natural language understanding, and topic segmentation, among others. In some embodiments, the query handler 145 may identify the selections on the user interface of the application running on the client device 115 for accessing the record service 105. With the identification of the selections, the query handler 145 may identify or determine the corresponding criteria for retrieval of records 165 from the aggregate record database 120.

With the identification from the query 180, the query handler 145 may access the aggregate record database 120 to find or identify a subset of records 165 that satisfy or match the one more criteria. In some embodiments, the query handler 145 may access the aggregate record database 120 to identify corresponding location identifiers to the records 165 that satisfy or match the criteria. In some embodiments, the query handler 145 may find the subset of records 165 from the aggregate record database 120 in accordance with the relational database management protocol used to maintain the aggregate record database 120. For example, the aggregate record database 120 may be maintained using SQL and the query 180 may also be generated using SQL. In this example, the query handler 145 may use the SQL LIKE operator to find the subset of records 165 from the aggregate record database 120 that match the criteria of the query 180. The subset identified using the query 180 may include records 165 or the location identifiers to the corresponding biomedical images 170 in the records 165, or a combination of both, depending on the format used by the data source 110 from which the record 165 originates. Furthermore, the subset of records 165 identified using the query 180 may include one or more files corresponding to the biomedical image 170 and the metadata 175 for each record 165. For example, the query handler 145 may identify one file containing the metadata 175 and one or more image files corresponding to the biomedical image 170. The query handler 145 may also find one or more image files corresponding to the biomedical image 170 with the metadata 175 embedded in the image files.

In some embodiments, the query handler 145 may traverse through the records 165 maintained on the aggregate record database 120 to compare with the criteria identified by the query 180. If the record 165 satisfies or matches the criteria, the query handler 145 may include the record 165 into the subset. In some embodiments, the query handler 145 may identify the location identifier for the record 165 (or the associated biomedical image 170) satisfying or matching the criterion to include into the subset. Conversely, if the record 165 does not satisfy or match the criteria, the query handler 145 may exclude the record 165 from the subset. In some embodiments, the query handler 145 may identify the number of subset of records 165 that match the remaining criteria as specified by the query 180. For example, if the query 180 specifies for 30 skin lesion histology slides, the query handler 145 may terminate the searching of the aggregate record database 120 upon finding 30 matching records 165.

In some embodiments, the query handler 145 may include or exclude the records 165 identified as satisfying or matching the criteria of the query 180 based on an indication of permission (sometimes referred herein as accession) for use. The indication of permission may, for example, correspond to a consent by the human subject from which the biological sample is obtained for the biological image 170 of the record 165. For each of the subset of records 165 satisfying or matching the criteria of the query 180, the query handler 145 may determine whether the indication of permission for use is present for the record 165. If the indication of the permission for use of the record 165 is determined to be present, the query handler 145 may maintain the record 165 in the subset identified using the query 180. Conversely, if the indication of the permission for use of the record 165 is determined to be not present, the query handler 145 may exclude the record 165 from the subset. The exclusion may be despite the record 165 satisfying or matching the selection criteria identified by the query 180.

For each record 165 identified using the query 180, the policy enforcer 150 running on the record service 105 may identify the data source 110 that generated the record 165. In some embodiments, the policy enforcer 150 may identify the label identifying the originating data source 110 for the record 165 on the aggregate record database 120. In some embodiments, in identifying the data source 110, the policy enforcer 150 may parse the record 165 (e.g., the one or more corresponding files) to identify the location identifier of the biomedical image 170. At least a portion of the location identifier may reference the data source 110, the associated data service 130, or the associated record database 135. Based on the referencing of the location identifier, the policy enforcer 150 may identify the data source 110 for the record 165.

Based on the identification of the data source 110 for the record 165, the policy enforcer 150 may identify or select a de-identification policy 160 from the set of de-identification policies 160 maintained by the record service 105 to apply to the record 165 in the subset. Each de-identification policy 160 may be particular or may correspond to one of the data sources 110 from which records 165 are gathered and maintained on the aggregate record database 120. The de-identification policy 160 selected by the policy enforcer 150 may correspond to that of the data source 110 from which the record 165 originates. In general, the de-identification policy may specify one or more operations to modify at least a portion of the metadata 175 from the record 165 generated in accordance with the format used by the originating data source 110. For example as illustrated in the following Table, the de-identification policy may specify:

| | |
|---|---|
| Original Metadata Type | De-Identified Metadata |
| Accession identifier | Case identifier |
| Accession date (mm/dd/year) | Accession year |
| Specimen class | No change |
| Part type | No change |
| Part instance | No change |
| Part description | De-identified |
| Block instance | No change |
| Block designator label | De-identified |
| Medical record number | Subject identifier |
| Slide image identifier | Image identifier |
| Scanning date (mm/dd/year) | Scan year |
| Stain type | No change |
| Synoptic data | No change |
| Subject trait | De-identified |
| Final diagnosis | De-identified diagnosis |

The operations specified by the de-identification policy 160 may include, for example, a truncation, a removal, or an overwrite of the portion of the metadata 175. The portions of the metadata 175 to be modified may also be specified by the de-identification policy 160. For example, the de-identification policy 160 may specify modification of metadata fields that originated from free text data entry (e.g. part description, block designator, and final diagnosis). The fields from free text data entry may be concatenated to a final report document stored as a plain text file. In accordance to the de-identification policy 160, the file may be redacted by replacing identifiers with placeholder text.

As the formats used to generate the records 165 differ from among the data sources 110, the de-identification policies 160 to be applied to the records 165 may vary depending on the data source 110 form which the corresponding record 165 originates. In some embodiments, the de-identification policy 160 may specify modification of the metadata 175 embedded into the biomedical image 170. For example, the de-identification policy 160 may specify at least one or more specified bytes in one or more image files constituting the biomedical image 170 to modify the metadata 175. The de-identification policy 160 may also specify onto one or more portions of the biomedical image 170 itself to modify the metadata 175. The de-identification policy 160 may also specify that the metadata 175 maintained the one or more files for the record 165 that are separate from the image files for the biomedical image 170 are to be modified. In some embodiments, the de-identification policy 160 may specify the retrieval of the one or more image files for the biomedical image 170 referenced by the corresponding location identifier, prior to modification of at least the portion of the metadata 175.

In accordance with the de-identification policy 160 selected for the record 165, the policy enforcer 150 may modify at least a portion of the metadata 175 identified by the record 165 to generate, derive, or otherwise obtain a de-identified record 165′. To modify, in some embodiments, the policy enforcer 150 may identify the one or more files corresponding to the record 165 as indicated by the de-identification policy 160. As discussed above, depending on the format used by the data source 110, the record 165 may correspond to at least one file containing the metadata 175 and one or more image files forming the biomedical image 710. The record 165 may also correspond to one or more image files corresponding to the biomedical image 170 with the metadata 175 embedded therein. For example, one image file for the biomedical image 170 may have at least one portion corresponding to the visual characteristics defining the rendering of the biomedical image 170 and at least one another portion corresponding to the metadata 175. What files are to be accessed to modify the metadata 175 may be specified by the de-identification policy 160 for the data source 110 from which the record 165 originates.

With the identification of the files, the policy enforcer 150 may parse each file to identify the portion to be modified as specified by the de-identified policy 160 selected for the record 165. When the de-identification policy 160 indicates that the metadata 175 are in the file separate from the image file, the policy enforcer 150 may access the file containing the metadata 175. With the file containing the metadata 175 accessed, the policy enforcer 150 may read the contents of the file to identify the one or more portion corresponding to the metadata 175 to be modified. Upon identification, the policy enforcer 150 may apply the one or more operations specified by the de-identification policy 160 to modify the metadata 175 (e.g., via removal, truncation, or overwrite). On the other hand, when the de-identification policy 160 indicates that the metadata 175 are included or embedded in the one or more image files, the policy enforcer 150 may access the image files corresponding to the biomedical image 170. In some embodiments, the policy enforcer 150 may identify the one or more portions in the accessed image files (e.g., bytes) corresponding to the metadata 175 embedded in the biomedical image 170. In some embodiments, the policy enforcer 150 may identify the one or more portion of the rendered image of the biomedical image 170 that contain the fields and values of the metadata 175. Based on the identifications, the policy enforcer 150 may modify the metadata 175 from the portions by applying the operations specified by the de-identification policy 160.

In conjunction with the application of the de-identification policy 160, the policy enforcer 150 may determine whether the record 165 include additional information to be modified by using one or more pattern recognition algorithms. The additional information may include protected health information (PHI) or other classified or sensitive information that remains subsequent to the application of the de-identification policy 160. For example, the full name of the subject from which the biological sample for the biomedical image 170 is acquired may appear elsewhere in the record 165, such as the final diagnosis field in the metadata 175 or somewhere on the rendering of the biomedical image 170. The pattern recognition algorithms may include, for example, a decision tree, support vector machine (SVM), an artificial neural network (ANN), an optical character recognition (OCR) algorithm, correlation clustering, discriminant analysis, and NLP techniques, among others. In determining, the policy enforcer 150 may apply the pattern recognition algorithm to the record 165, such as file containing the metadata 175, the image files forming the biomedical image 170, the rendered image corresponding to the biomedical image 170, or any combination thereof. When the record 165 is determined to not include any additional information using the pattern recognition algorithms, the policy enforcer 150 may maintain the record 165 as is in the subset. Conversely, when the record is determined to include the additional information using the pattern recognition algorithms, the policy enforcer 150 may recognize or identify one or more portions in the record 165 corresponding to the additional information. With the identification, the policy enforcer 150 may modify (e.g., remove, truncate, or overwrite) the additional information in the record 165 to obtain the de-identified record 165′.

The cohort packager 155 running on the record service 105 may transmit, send, or provide the de-identified records 165′ obtained from the subset of records 165 that is identified using the query 180 to the client device 115. In some embodiments, the provision of the de-identified records 165′ may be responsive to the applications of the corresponding de-identification policies 160 or the pattern recognition algorithms, or both. With the obtaining the de-identified records 165′, the cohort packager 155 may combine, join, or otherwise package the de-identified records 165′ into a record set (also sometimes referred herein as a cohort) to provide as a response to the client device 115. Once packaged, the cohort packager 155 may send or provide the record set to the client device 115 via the network 125 or 125′.

Furthermore, the cohort packager 155 may store and maintain the de-identified records 165′ onto the aggregate record database 120. In storing, for each de-identified record 165′, the cohort packager 155 may identify the original record 165 corresponding to the de-identified record 165′. Using the identification, the cohort packager 155 may associate or link the original record 165 with the corresponding, de-identified record 165′. In some embodiments, the cohort packager 155 may generate or include a label identifying the corresponding record 165 in the de-identified record 165′, or vice-versa. The cohort packager 155 may store and maintain the association or link between the original record 165 and the de-identified record 165′. The de-identified record 165′ may be found and identified using subsequent queries 180 from one or more of the client devices 115. For example, to avoid applying the de-identification policy 160 to the record 165 identified using the query 180, the query handler 145 may identify the de-identified record 165′ corresponding to the record 165.

In this manner, the application of the computationally complex application of the de-identification policy 160 on the records 165 (with biomedical images 170 ranging in 500 megabytes to 5 gigabytes) may be reduced or limited to on-demand requests (e.g., upon receipt of the query 180 from the client device 115). Since the repeated applications of the de-identification policy 160 is reduced, the consumption of computing resources by the record service 105 may be reduced or decreased, thereby freeing up the record service 105 to perform other processes and tasks. Furthermore, queries 180 for records 165 from multiple data sources 110 may be processed at a centralized location, thereby avoiding the client device 115 from sending multiple requests to different data sources 110.

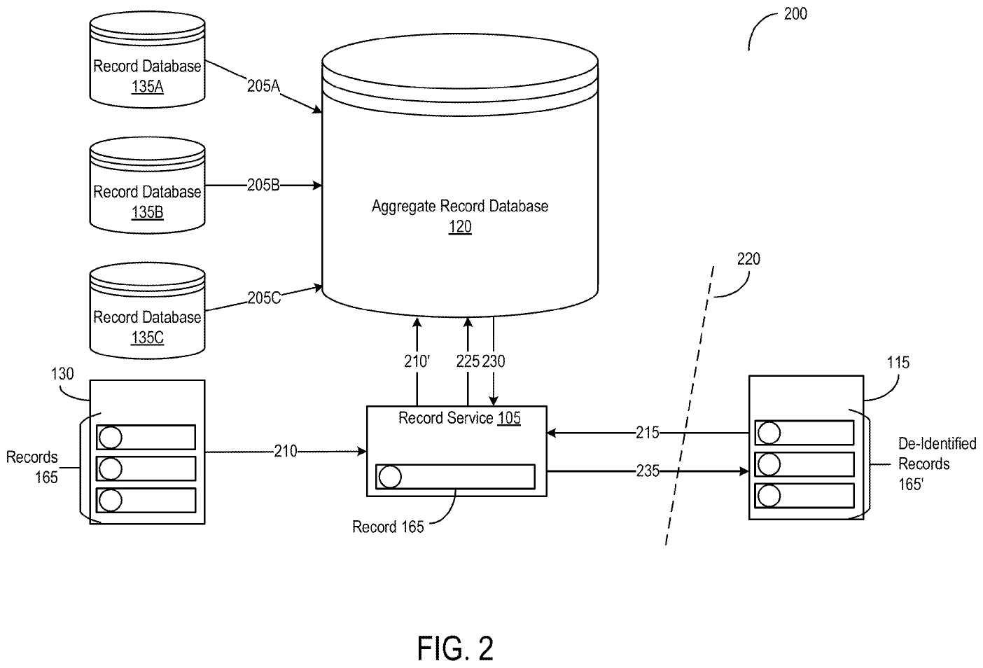

Referring now to FIG. 2, depicted is a sequence diagram of an example process 200 for maintaining databases of biomedical images. Under process 200, the aggregate record database 120 may pull and aggregate records 165 from multiple record databases 135. The records 165 from the first record database 135A (e.g., an image management system) may be aggregated via communication 205A. The records 165 from the second record database 135B (e.g., a laboratory information system) may be aggregated via communication 205B. The records 165 from the third record database 135C (e.g., institutional database) may be aggregated via communication 205C. In addition, the record service 105 may pull and receive records 165 from one of the data services 130 (e.g., a slide archive server) via communication 210 and store onto the aggregate record database 120 via communication 210′.

In conjunction, the record service 105 may receive the query 180 from one of the client devices 115 via communication 215. The query 180 sent by the client device 115 via the communication 215 may traverse at least one network access control 220 (e.g., a network firewall, authorization, or authentication) between the record service 105 and the client device 115. The network access control 220 may be formed from having two separate networks to communicate with the record service 105, with the network 125 for communications between the record service 105 and the client device 115 and the network 125′ for communications among the record service 105 and various data sources 110. Upon receipt, the record service 105 may access the aggregate record database 120 to search for records 165 satisfying the query 180 via communication 225. The record service 105 may retrieve or fetch the records 165 matching the query 180 via communication 230. Upon finding the records 165, the record service 105 may apply the respective de-identification policies 160 and provide the de-identified records 165′ via communication 235 through the network access control 220.

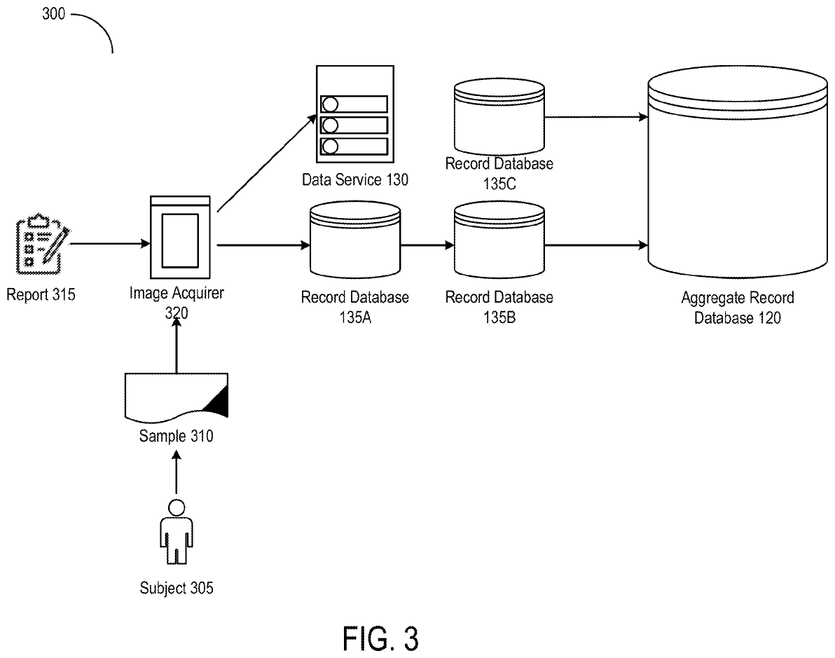

Referring now to FIG. 3, depicted is a sequence diagram of an example process 300 for maintaining databases of biomedical image. Under process 300, a subject 305 may be provide at least one biological sample 310, sections of which may be placed on slide. The subject 305 may have provided consent to take the biological sample 310 for use in research. Separately, a report 315 may be created via inputs on a computing device by a clinician examining the biological sample 310. The report 315 may correspond to fields and values for the metadata 175 associated with the subject 305 or the biological sample 310. An image acquirer 320 (e.g., a computing device communicatively coupled with a microscopy camera) may acquire an image of the sample 310 to generate a biomedical image 170 (e.g., in the form of one or more image files). In addition, the image acquirer 320 may combine or associate the biomedical image 170 with the report 315 in accordance with the format used by the data source 110 associated with the image acquirer 320 to generate a record 165.

The record 165 generated using the format may be stored on the data service 130 itself or the first record database 135A of the same data source 110. For example, the biomedical image 170 may be stored on the data service 130 and the metadata 175 for the biomedical image 170 may be stored onto the first record database 135A (e.g., an image management system). The metadata 175 along with the location identifier for the biomedical image 170 may be forwarded or sent to the second record database 135B (e.g., a laboratory information system). In conjunction, an indication of the permission for use (e.g., accession or consent by the subject 305) by the subject 305 may be stored onto the third record database 135C (e.g., the institutional database). The record 165 may be gathered and maintained onto the aggregate record database 120 from the record databases 135A-C and the data service 130. For example, the location identifier for the biomedical image 170 and the metadata 175 for the record 165 may be fetched from the second record database 135B. The indication of the permission for use may be pulled from the third record database 135C.

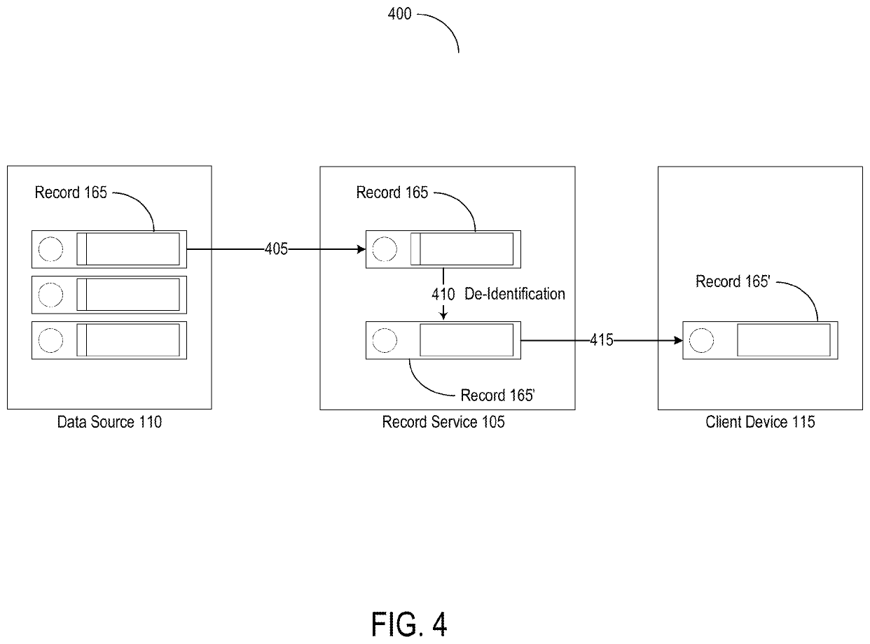

Referring now to FIG. 4, depicted is a sequence diagram of an example process 400 for maintaining databases of biomedical images. Under the process 400, one of the records 165 of the data source 110 may be identified as satisfying the criteria of a query 180 from the client device 115, and may be provided to the record service 105 via communication 405. Each record 165 may have the biomedical image 170 and the metadata 175 packaged according to the format used by the data source 110. Upon receipt, the record service 105 may perform de-identification 410 to the record 165 in accordance with the de-identification policy 160 for the data source 110 that generated the record 165. With the application of the de-identification policy 160, the record service 105 may obtain the de-identified record 165′. The record service 105 may provide the de-identified record 165′ to the client device 115 via the communication 415.

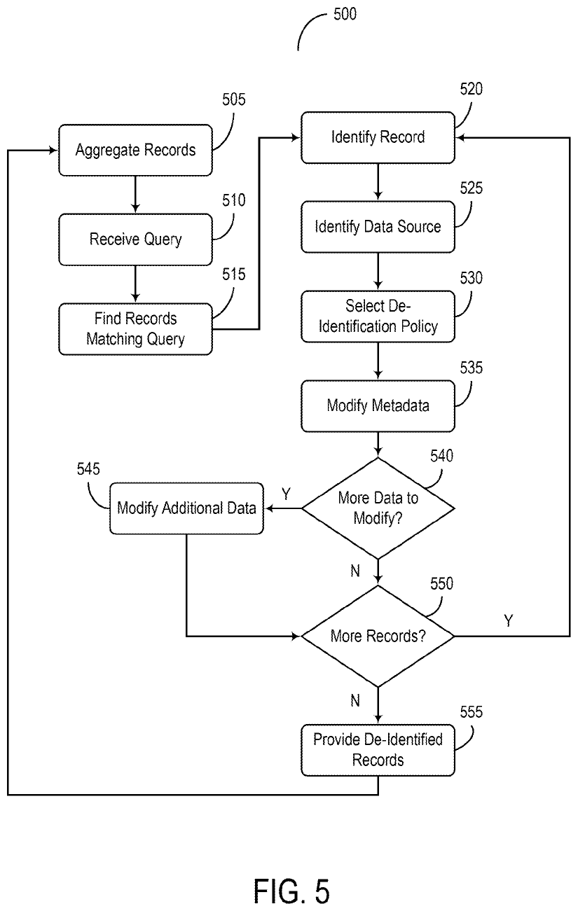

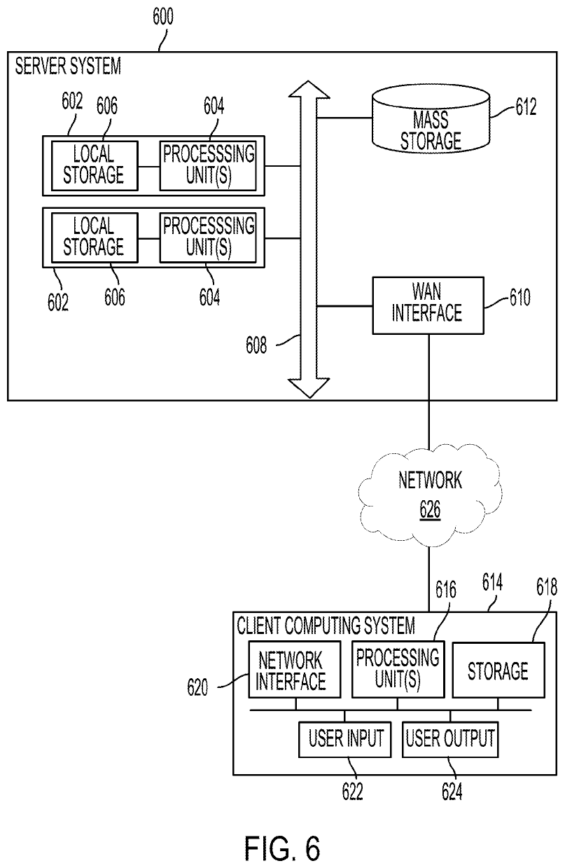

Referring now to FIG. 5, depicted is a flow diagram of a method 500 of maintaining databases of biomedical images. The method 500 may be implemented using or performed by any of the components in the system 100 as detailed herein in conjunction with FIGS. 1-4 or the computing system 600 as described herein in conjunction with FIG. 6. In overview, in method 500, a record service (e.g., the record service 105) may aggregate digital pathology records (e.g., the records 165) (505). The record service may receive a query (e.g., the query 180) (510). The record service may find digital pathology records matching the query (515). The record service may identify a digital pathology record (520). The record service may identify a data source (e.g., the data source 110) of the digital pathology record (525). The record service may select a de-identification policy (e.g., the de-identification policy 160) for the data source (530). The record service may modify metadata (e.g., the metadata 175) in accordance with the de-identification policy (535). The record service may determine whether there is more data to modify (540). If there is more data to modify, the record service may modify the additional data (545). In any event, the record service may determine whether there are more digital pathology records (550). If there are more digital pathology records, the functionality of (520)—(545) may be repeated. Otherwise, if there are no more digital pathology records, the record service may provide de-identified digital pathology records (e.g., the de-identified records 165′) (555).

### B. Computing and Network Environment

Various operations described herein can be implemented on computer systems. FIG. 6 shows a simplified block diagram of a representative server system 600, client computer system 614, and network 626 usable to implement certain embodiments of the present disclosure. In various embodiments, server system 600 or similar systems can implement services or servers described herein or portions thereof. Client computer system 614 or similar systems can implement clients described herein. The system 100 described herein can be similar to the server system 600. Server system 600 can have a modular design that incorporates a number of modules 602 (e.g., blades in a blade server embodiment); while two modules 602 are shown, any number can be provided. Each module 602 can include processing unit(s) 604 and local storage 606.

Processing unit(s) 604 can include a single processor, which can have one or more cores, or multiple processors. In some embodiments, processing unit(s) 604 can include a general-purpose primary processor as well as one or more special-purpose co-processors such as graphics processors, digital signal processors, or the like. In some embodiments, some or all processing units 604 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 604 can execute instructions stored in local storage 606. Any type of processors in any combination can be included in processing unit(s) 604.

Local storage 606 can include volatile storage media (e.g., DRAM, SRAM, SDRAM, or the like) and/or non-volatile storage media (e.g., magnetic or optical disk, flash memory, or the like). Storage media incorporated in local storage 606 can be fixed, removable or upgradeable as desired. Local storage 606 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 604 need at runtime. The ROM can store static data and instructions that are needed by processing unit(s) 604. The permanent storage device can be a non-volatile read-and-write memory device that can store instructions and data even when module 602 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 606 can store one or more software programs to be executed by processing unit(s) 604, such as an operating system and/or programs implementing various server functions such as functions of the system 100 of FIG. 1 or any other system described herein, or any other server(s) associated with system 100 or any other system described herein.

“Software” refers generally to sequences of instructions that, when executed by processing unit(s) 604 cause server system 600 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 604. Software can be implemented as a single program or a collection of separate programs or program modules that interact as desired. From local storage 606 (or non-local storage described below), processing unit(s) 604 can retrieve program instructions to execute and data to process in order to execute various operations described above.

In some server systems 600, multiple modules 602 can be interconnected via a bus or other interconnect 608, forming a local area network that supports communication between modules 602 and other components of server system 600. Interconnect 608 can be implemented using various technologies including server racks, hubs, routers, etc.

A wide area network (WAN) interface 610 can provide data communication capability between the local area network (interconnect 608) and the network 626, such as the Internet. Technologies can be used, including wired (e.g., Ethernet, IEEE 802.3 standards) and/or wireless technologies (e.g., Wi-Fi, IEEE 802.11 standards).

In some embodiments, local storage 606 is intended to provide working memory for processing unit(s) 604, providing fast access to programs and/or data to be processed while reducing traffic on interconnect 608. Storage for larger quantities of data can be provided on the local area network by one or more mass storage subsystems 612 that can be connected to interconnect 608. Mass storage subsystem 612 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 612. In some embodiments, additional data storage resources may be accessible via WAN interface 610 (potentially with increased latency).

Server system 600 can operate in response to requests received via WAN interface 610. For example, one of modules 602 can implement a supervisory function and assign discrete tasks to other modules 602 in response to received requests. Work allocation techniques can be used. As requests are processed, results can be returned to the requester via WAN interface 610. Such operation can generally be automated. Further, in some embodiments, WAN interface 610 can connect multiple server systems 600 to each other, providing scalable systems capable of managing high volumes of activity. Other techniques for managing server systems and server farms (collections of server systems that cooperate) can be used, including dynamic resource allocation and reallocation.

Server system 600 can interact with various user-owned or user-operated devices via a wide-area network such as the Internet. An example of a user-operated device is shown in FIG. 6 as client computing system 614. Client computing system 614 can be implemented, for example, as a consumer device such as a smartphone, other mobile phone, tablet computer, wearable computing device (e.g., smart watch, eyeglasses), desktop computer, laptop computer, and so on.

For example, client computing system 614 can communicate via WAN interface 610. Client computing system 614 can include computer components such as processing unit(s) 616, storage device 618, network interface 620, user input device 622, and user output device 624. Client computing system 614 can be a computing device implemented in a variety of form factors, such as a desktop computer, laptop computer, tablet computer, smartphone, other mobile computing device, wearable computing device, or the like.

Processor 616 and storage device 618 can be similar to processing unit(s) 604 and local storage 606 described above. Suitable devices can be selected based on the demands to be placed on client computing system 614; for example, client computing system 614 can be implemented as a “thin” client with limited processing capability or as a high-powered computing device. Client computing system 614 can be provisioned with program code executable by processing unit(s) 616 to enable various interactions with server system 600.

Network interface 620 can provide a connection to the network 626, such as a wide area network (e.g., the Internet) to which WAN interface 610 of server system 600 is also connected. In various embodiments, network interface 620 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 622 can include any device (or devices) via which a user can provide signals to client computing system 614; client computing system 614 can interpret the signals as indicative of particular user requests or information. In various embodiments, user input device 622 can include any or all of a keyboard, touch pad, touch screen, mouse or other pointing device, scroll wheel, click wheel, dial, button, switch, keypad, microphone, and so on.

User output device 624 can include any device via which client computing system 614 can provide information to a user. For example, user output device 624 can include a display to display images generated by or delivered to client computing system 614. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 624 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 604 and 616 can provide various functionality for server system 600 and client computing system 614, including any of the functionality described herein as being performed by a server or client, or other functionality.

It will be appreciated that server system 600 and client computing system 614 are illustrative and that variations and modifications are possible. Computer systems used in connection with embodiments of the present disclosure can have other capabilities not specifically described here. Further, while server system 600 and client computing system 614 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies including but not limited to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method of maintaining databases of biomedical images, comprising:
aggregating, by one or more processors, a plurality of digital pathology records from a plurality of data sources onto a database, each of the plurality of digital pathology records generated by a data source of the plurality of data sources in accordance with a format used by the data source, each of the plurality of digital pathology records identifying a biomedical image of a sample and data identifying a subject from which the sample is obtained;
receiving, by the one or more processors from a client device, a query identifying a selection criterion for retrieving digital pathology records from the database;
accessing, by the one or more processors, the database to identify a subset of digital pathology records from the plurality of digital pathology records using the selection criterion identified by the query;
for each digital pathology record of the subset:
identifying, by the one or more processors, a data source of the plurality of data source that generated the digital pathology record;
selecting, by the one or more processors, from a plurality of de-identification policies, a de-identification policy to apply to the digital pathology record based on the data source;
modifying, by the one or more processors, the data identifying the subject from the digital pathology record in accordance with the selected de-identification policy and the format used by the data source to obtain a de-identified digital pathology record; and
providing, by the one or more processors to the client device, the de-identified digital pathology record in response to modifying the data identified the subject.

2. The method of claim 1, further comprising identifying, by the one or more processors for each digital pathology record of the subset, in accordance with the de-identification policy, the data to be modified in the digital pathology record, the de-identification specifying at least one of a truncation, a removal, or an overwrite of at least a corresponding portion of the data.

3. The method of claim 1, further comprising for at least one digital pathology record of the subset:
identifying, by the one or more processors, using pattern recognition, additional information to modify from the digital pathology record subsequent to modifying the data in accordance with the de-identification policy; and
modifying, by the one or more processors, the additional information in the digital pathology record to obtain the de-identified digital pathology record.

4. The method of claim 1, further comprising identifying, by the one or more processors for at least one digital pathology record of the subset, a first file containing the data and a second file containing the biomedical image for the digital pathology record in accordance with the format used by the data source to generate the digital pathology record; and
wherein modifying the data further comprises modifying the data contained in the first file separate from the second file in accordance with the de-identification policy.

5. The method of claim 1, further comprising identifying, by the one or more processors for at least one digital pathology record of the subset, a file including a first portion corresponding to the data and one or more second portions corresponding to the biomedical image for the digital pathology record in accordance with the format used by the data source to generate the digital pathology record; and
wherein modifying the data further comprises modifying the data in the first portion of the file for the digital pathology record of the subset in accordance with the de-identification policy.

6. The method of claim 1, wherein aggregating the plurality of digital pathology records further comprises aggregating a plurality of location identifiers from the plurality of data sources, the plurality of location identifiers identifying the biomedical image and the data for each of the plurality of digital pathology records, and
wherein accessing the database further comprises retrieving the subset of digital pathology records from one or more of the plurality of data sources using a subset of location identifiers corresponding to the subset of digital pathology records.

7. The method of claim 1, wherein accessing the database further comprises accessing the database to identify the subset of digital pathology records from the plurality of digital pathology records, each of the subset of digital pathology records having an indication of permission for use.

8. The method of claim 1, wherein aggregating the plurality of digital pathology records further comprising maintaining the plurality of digital pathology records retrieved from the plurality of data sources, without removal of the data identifying the subject in each of the plurality of digital pathology records prior to receiving the query.

9. The method of claim 1, wherein aggregating the plurality of digital pathology records further comprises aggregating the plurality of digital pathology records, each of the plurality of digital pathology records identifying the data identifying a date at which the biomedical image of the sample from the subject is acquired, a part description, an image identifier, and a descriptor.

10. The method of claim 1, further comprising storing, by the one or more processors, for each digital pathology record of the subject, the de-identified digital pathology record onto the database to replace the corresponding digital pathology record of the subject.

11. A system for maintaining databases of biomedical images, comprising:
one or more processors coupled with memory, configured to:
aggregate a plurality of digital pathology records from a plurality of data sources onto a database, each of the plurality of digital pathology records generated by a data source of the plurality of data sources in accordance with a format used by the data source, each of the plurality of digital pathology records identifying a biomedical image of a sample and data identifying a subject from which the sample is obtained;
receive, from a client device, a query identifying a selection criterion for retrieving digital pathology records from the database;
access the database to identify a subset of digital pathology records from the plurality of digital pathology records using the selection criterion identified by the query;
for each digital pathology record of the subset:
identify a data source of the plurality of data source that generated the digital pathology record;
select, from a plurality of de-identification policies, a de-identification policy to apply to the digital pathology record based on the data source;
modify the data identifying the subject from the digital pathology record in accordance with the selected de-identification policy and the format used by the data source to obtain a de-identified digital pathology record; and
provide, to the client device, the de-identified digital pathology record in response to modifying the data identified the subject.

12. The system of claim 11, wherein the one or more processors are further configured to identify, for each digital pathology record of the subset, in accordance with the de-identification policy, the data to be modified in the digital pathology record, the de-identification specifying at least one of a truncation, a removal, or an overwrite of at least a corresponding portion of the data.

13. The system of claim 11, wherein the one or more processors are further configured to, for at least one digital pathology record of the subset:
identify, using pattern recognition, additional information to modify from the digital pathology record subsequent to modifying the data in accordance with the de-identification policy; and
modify the additional information in the digital pathology record to obtain the de-identified digital pathology record.

14. The system of claim 11, wherein the one or more processors are further configured to:
identify, for at least one digital pathology record of the subset, a first file containing the data and a second file containing the biomedical image for the digital pathology record in accordance with the format used by the data source to generate the digital pathology record; and
modify the data contained in the first file separate from the second file in accordance with the de-identification policy.

15. The system of claim 11, wherein the one or more processors are further configured to:
identify, for at least one digital pathology record of the subset, a file including a first portion corresponding to the data and one or more second portions corresponding to the biomedical image for the digital pathology record in accordance with the format used by the data source to generate the digital pathology record; and
modify the data in the first portion of the file for the digital pathology record of the subset in accordance with the de-identification policy.

16. The system of claim 11, wherein the one or more processors are further configured to:
aggregate a plurality of location identifiers from the plurality of data sources, the plurality of location identifiers identifying the biomedical image and the data for each of the plurality of digital pathology records, and
retrieve the subset of digital pathology records from one or more of the plurality of data sources using a subset of location identifiers corresponding to the subset of digital pathology records.

17. The system of claim 11, wherein the one or more processors are further configured to access the database to identify the subset of digital pathology records from the plurality of digital pathology records, each of the subset of digital pathology records having an indication of permission for use.

18. The system of claim 11, wherein the one or more processors are further configured to maintain the plurality of digital pathology records retrieved from the plurality of data sources, without removal of the data identifying the subject in each of the plurality of digital pathology records prior to receiving the query.

19. The system of claim 11, wherein the one or more processors are further configured to aggregate the plurality of digital pathology records, each of the plurality of digital pathology records identifying the data identifying a date at which the biomedical image of the sample from the subject is acquired, a part description, an image identifier, and a descriptor.

20. The system of claim 11, wherein the one or more processors are further configured to store, aggregating the plurality of digital pathology records, each of the plurality of digital pathology records identifying the data identifying a date at which the biomedical image of the sample from the subject is acquired, a part description, an image identifier, and a descriptor.
